(19) United States
(12) Patent Application Publication
(10) Pub. No.: US 2005/0246007 A1
(43) Pub. Date: Nov. 3, 2005

(54) Novel lead body assemblies

(75) Inventors: John L. Sommer, Coon Rapids, MN (US); Douglas N. Hess, Maple Grove, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/833,511

(22) Filed: Apr. 28, 2004

(51) Int. Cl. A61N 1/05

(52) U.S. Cl. 607/127

(57) ABSTRACT

A medical electrical lead comprises an elongate conductor and a sheath extending over the conductor to form a rotating assembly.

### TECHNICAL FIELD

The present invention relates to medical electrical leads and more particularly to novel lead body assemblies.

### BACKGROUND

Cardiac stimulation systems commonly include a pulse-generating device, such as a pacemaker or implantable cardioverter/defibrillator that is electrically connected to the heart by at least one electrical lead. An electrical lead delivers electrical pulses emitted by the pulse generator to the heart, stimulating the myocardial tissue via electrodes included on the lead. Furthermore, cardiac signals may be sensed by lead electrodes and conducted, via the lead, back to the device, which also monitors the electrical activity of the heart.

Medical electrical leads are typically constructed to have the lowest possible profile without compromising functional integrity, reliability and durability. One aspect of lead function includes fixation at an implant site and one category of leads includes those that employ active fixation mechanisms; one type of active fixation mechanism known in the art is an extendable-retractable screw or helix. The helix is housed in proximity to a distal tip of the lead and is coupled to an elongate coil extending proximally from the helix within a body of the lead to a proximal end of the lead where the coil is coupled to a connector pin; the connector pin is rotated in one direction to extend the helix out from the housing and in an opposite direction to retract the helix back into the housing. Another type of active fixation lead known in the art employs a fixed screw permanently extended from a distal tip of the lead and wherein the body of the lead is rotated to fix the screw into an implant site. For either type of active fixation mechanism, it is desirable to employ a coil that has torque-transfer capability approaching a 1:1 ratio of connector pin turns to helix turns; however additional requirements on the overall lead design, for example electrical inductance, electrical resistance and outer diameter, can dictate coil characteristics, which may conflict with this desire.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a plan view with a partial cut-away view of an exemplary medical electrical lead in which embodiments of the present invention may be incorporated;

FIG. 2 is a section view of a distal portion of the lead shown in FIG. 1 according to one embodiment of the present invention;

FIG. 3 is a section view of a proximal portion of the lead shown in FIG. 2 according to one embodiment of the present invention;

FIG. 4 is a section view of a portion of a body of the lead shown in FIG. 1 according to an embodiment of the present invention;

FIG. 5 is a section view of a distal or proximal portion of a lead according to an alternate embodiment of the present invention;

FIG. 6 is a perspective section view of a lead body according to another embodiment of the present invention; and

FIG. 7 is a plan view with partial cut-away views of an exemplary medical electrical lead according to yet another embodiment of the present invention.

### DETAILED DESCRIPTION

The following detailed description is exemplary in nature and is not intended to limit the scope, applicability, or configuration of the invention in any way. Rather, the following description provides a practical illustration for implementing exemplary embodiments of the invention.

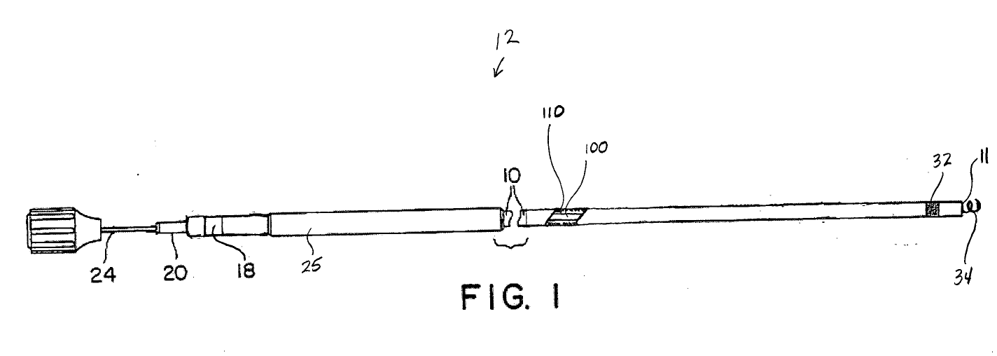

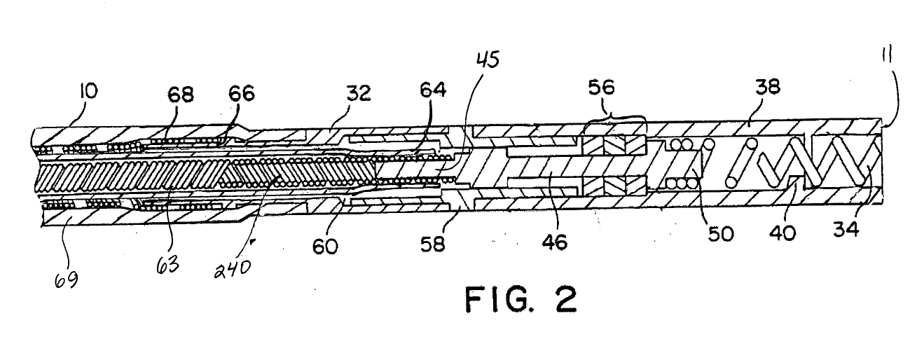

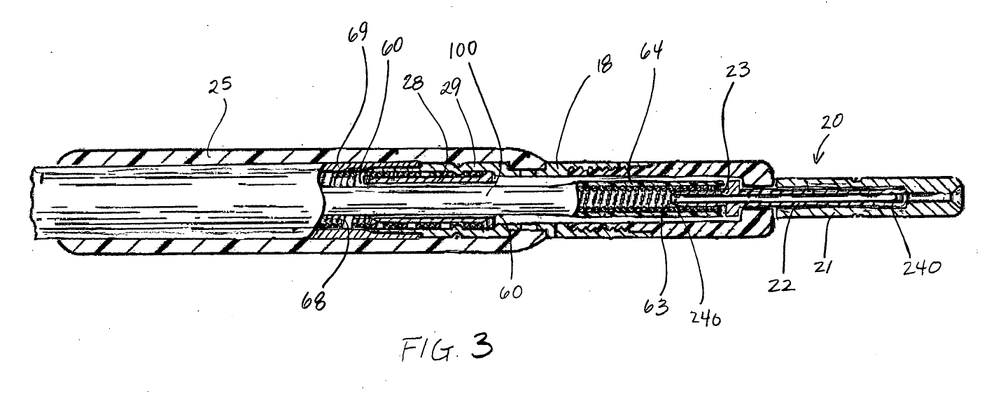

FIG. 1 is a plan view with a partial cut-away view of an exemplary medical electrical lead in which embodiments of the present invention may be incorporated. FIG. 1 illustrates an elongate body 10 of a lead 12 including an outer assembly 110 and an inner assembly 100 extending within outer assembly 110; body 10 extends from a proximal end, which includes a connector pin 20, a connector ring 18 and a connector sleeve 25 to a distal end, which includes a ring electrode 32 and a helix 34, which may include an electrode surface. The proximal end of lead 12 is adapted for coupling with a pulse generator and the distal end adapted for implantation in a body. According to embodiments of the present invention an rotating assembly, which is adapted to rotate within outer assembly 110 to extend and retract helix 34, includes connector pin 20, inner assembly 100 and helix 34. FIG. 1 further illustrates a stylet wire 24 inserted within an inner lumen of lead body 10, that is lumen 240 defined by connector pin 20 and an inner coil 63 shown in FIGS. 2 and 3; stylet wire 24 may be used to deliver lead 12 to an implant site according to methods well known to those skilled in the art.

FIG. 2 is a section view of a distal portion of the lead shown in FIG. 1; and FIG. 3 is a section view of a proximal portion of the lead shown in FIG. 2. FIGS. 2 and 3 illustrate outer assembly 110 including an outer insulative sheath 69 surrounding an outer conductor coil 68, which surrounds inner assembly 100 that includes an inner insulative sheath 64 surrounding an inner conductor coil 63. FIGS. 2 and 3 further illustrate outer conductor 68 coupled, at a distal end, to ring electrode 32 and, at a proximal end, to connector ring 18; and inner conductor 63 coupled, at a distal end, to helix 34 via a helix stud 46 and, at a proximal end to connector pin 20. Any appropriate coupling means for inner and outer conductors 63, 68 that is known to those skilled in the art, for example crimping and welding, may be employed.

FIG. 2 illustrates ring electrode 32, which is another part of outer assembly 100, including an extension 66 to which outer coil 68 is coupled. FIG. 2 further illustrates helix stud 46 extending through a seal assembly 56 and including a stud proximal end 45 to which inner coil 63 is coupled and a stud distal end 50 to which helix 34 is coupled. In FIG. 2, helix 34 is illustrated in a retracted position housed within a sleeve-head 38, which is yet another part of outer assembly 110 and is coupled to ring electrode 32 via interfacing component 58. According to the illustrated embodiment, helix 34 would function as an electrode, however according to an alternate embodiment, helix 34 only serves for fixation and an electrode is coupled to a distal tip 11 and configured for electrical coupling with conductor 63, for example via stud 46 when helix 34 is extended. FIG. 3 illustrates an extension 29 of connector ring 18, which is another part of outer assembly 100, crimped to a proximal end of outer coil 68, which is fitted over a sleeve 28 for support. FIG. 3 further illustrates connector pin 20 including a pin cap 21 coupled to a pin core 22, which extends into the outer assembly; inner coil 63 is mounted on an internal extension 23 of pin core 22 for coupling.

According to one embodiment of the present invention, inner coil 63 and inner sheath 64 are both fixedly coupled to connector pin 20, as illustrated in FIG. 3 at internal extension 23 of pin core 22, and to helix 34, as illustrated in FIG. 2 at stud proximal end 45, such that inner sheath 64 is an integral part of the rotating assembly which rotates within outer assembly 110 to extend and retract helix 34 out from and into sleeve-head 38. Inner coil 63 may be formed of any appropriate conductive material, an example of which is MP35N, and inner sheath 64 may be formed of any appropriate biocompatible material, examples of which include silicones, polyurethanes, polyimides and fluoropolymers. Inner sheath 64 may be fixedly coupled to the rotating assembly by means of an interference-fit between inner sheath 64 and coil 63 and/or one or both of stud proximal end 45 and internal extension 29, embedment of coil 63 within a wall of sheath 64, or adhesive filling and/or bonding between sheath 64 and/or one or both of stud proximal end 45 and internal extension 29. By including sheath 64 as an integral part of the extendable retractable assembly an inner conductor, i.e. coil 63, may be designed to meet requirements other than efficient torque transfer.

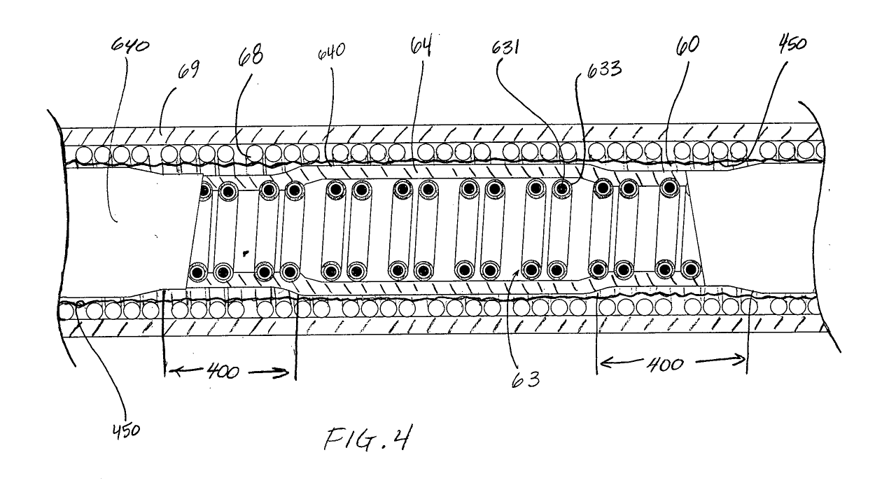

FIG. 4 is a section view of a portion of a body of the lead shown in FIG. 1 according to an embodiment of the present invention wherein inner sheath 64 is fixedly coupled to inner coil 63 by embedment of coil 63 in wall of sheath 64 along intermittent lengths 400; embedment may alternately be described as a mechanical interlocking between sheath 64 and coil 63. One example of the embodiment illustrated in FIG. 4 includes sheath 64 having been formed of a polyurethane tube that is fitted over coil 63 and then heat re-flowed into interstices between coil filars over lengths 400. According to alternate embodiments length 400 extends either along a longer length of a proximal, central or distal portion of coil 63, or along approximately an entire length of coil 63. According to one exemplary method, a polyurethane tube as sheath 64 is fitted about coil 63 and then another tube of silicone rubber is swelled in heptane and assembled over an entire length or a limited length of sheath 63; once the heptane has evaporated, the silicone rubber tube forms an interference fit about sheath 64 and coil 63, for example a 0.003 inch to 0.005 inch interference fit. The silicone tube provides a compressive force that facilitates uniform re-flow of the polyurethane when a temperature, for example between approximately 325° and 340° Fahrenheit, is applied to selected zones along the assembly or along an entire length; after the polyurethane is re-flowed to embed the underlying coil, the silicone tubing is removed.

FIG. 4 further illustrates a layer 450 positioned between outer coil 68 and inner sheath 64; according to one embodiment layer 450 is a lubricious interface facilitating rotation of the rotating assembly, which includes sheath 64 and inner coil 63, within outer assembly 110 (FIG. 1). Layer 450 may be an independent component, such as a liner, inserted in the space between the assemblies or may be a coating or a surface treatment of either an outer surface 640 of sheath 64 or an inner surface of coil 68; examples of appropriate materials for layer 450 include fluoropolymers and polyacrylamides known to those skilled in the art. According to alternate embodiments of the present invention inner sheath 64 may be formed of a conductive polymer, examples of which include intrinsically conductive polymers, such as polyacetylene and polypyrrole, and conductor-filled polymers, such as silicone rubber having embedded metallic, carbon, or graphite particles; in this case, layer 450 serves to electrically isolate the rotating assembly from conductor coil 68.

FIG. 4 further illustrates inner coil 63 having a bifilar construction, each filar being formed of a conductor wire 631 including a low resistance core and having an insulative coating 633, for example a polyimide or fluoropolymer coating. According to an exemplary embodiment of the present invention, coil 63 has an outer diameter of less than approximately 0.03 inch and conductor wire 631 is formed of silver-cored MP35N having an outer diameter of less than or equal to approximately 0.006 inch; sheath 64 being coupled to coil 63 may enhance torque transfer of coil 63 which may otherwise be insufficient to extend helix 34 without an excessive number of connector pin 20 turns.

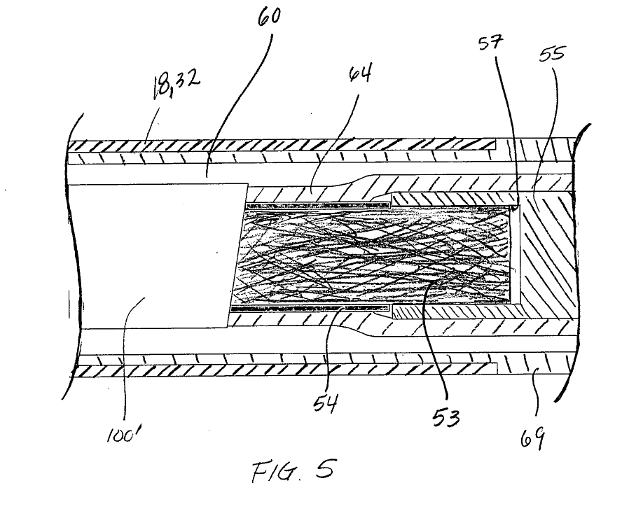

It should be noted that although FIGS. 1-4 illustrate outer assembly 110 including conductor 68, connector ring 18 and ring electrode 32 alternate embodiments may be unipolar rather than bipolar, that is outer assembly 110 may not include an additional electrical circuit formed by these elements. Furthermore inner assembly 100 may include a cabled bundle of conductor wires rather than coil 63 as will be described in conjunction with FIG. 5.

FIG. 5 is a section view of a distal or proximal portion of a lead according to an alternate embodiment of the present invention wherein a cable conductor 53 is employed as an inner conductor. FIG. 5 illustrates an inner assembly 100′ including a cable conductor 53 having an insulative coating 54 extending within inner sheath 64; both conductor 53 and sheath 64 are shown fixedly coupled to a junction element 55, which may either couple to an extendable retractable fixation element, for example helix 34, or to a connector pin, for example pin 20, depending on whether we view FIG. 5 as the distal portion or the proximal portion of the lead. According to some embodiments of the present invention cable 53 is crimped within junction element 55 and sheath 64 is mounted about junction element 55 such that sheath 64 is an integral part of the rotating assembly previously described; according to other embodiments sheath 64 is fixedly coupled along a length of cable 53 and may form a part of insulative coating 54 or be attached to insulative coating 54.

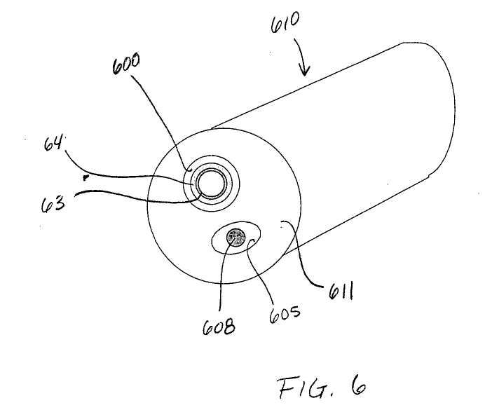

FIG. 6 is a perspective section view of a lead body 610 according to another embodiment of the present invention. FIG. 6 illustrates an outer insulative sheath in the form of a multi-lumen tube 611 including a first lumen 600 carrying inner coil 63 and inner sheath 64 of the rotating assembly and a second lumen 605 carrying a second conductor 608 which would be coupled at a proximal end to connector ring 18 and at a distal end to ring electrode 32. According to the illustrated embodiment, sheath 64 is an integral part of the rotating assembly, which rotates within first lumen 600; alternate means for coupling sheath 64 to the assembly are illustrated in FIGS. 2-4. Although FIGS. 2 and 3 illustrate a coaxial assembly, means for implementing similar distal and proximal couplings of coil 63 and sheath 64 to helix 34 and connector pin 20, respectively, and means to implement distal and proximal couplings of conductor 608 to ring electrode 32 and connector ring 18, respectively, are well known to those skilled in the art.

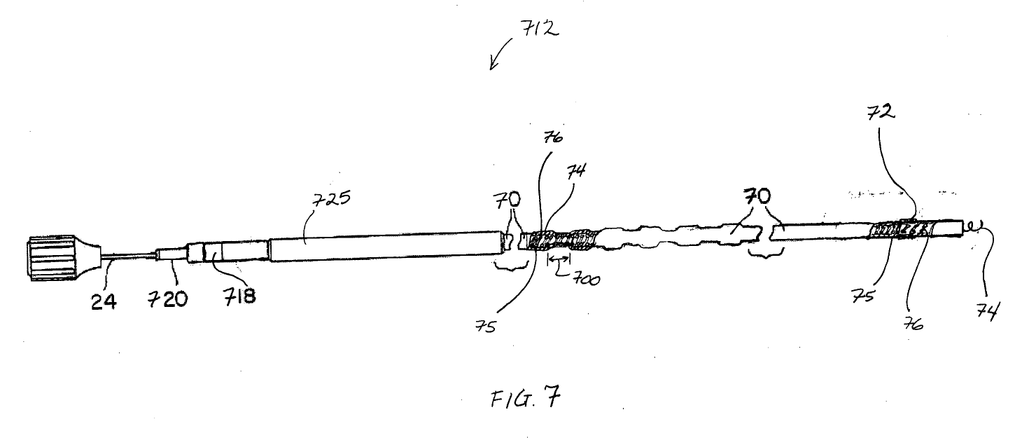

FIG. 7 is a plan view with partial cut-away views of an exemplary active fixation medical electrical lead, which employs a fixed screw, according to yet another embodiment of the present invention. FIG. 7 illustrates an elongate body 70 of a lead 712 including an outer sheath 74 and a conductor coil including a first filar 75 and a second filar 76, which are embedded in sheath 74 along intermittent lengths 700 such that sheath 74 is fixedly coupled to the coil. According to the illustrated embodiment, first filar 75 is coupled to a connector ring 718 at one end and to a ring electrode 72 at another end while second filar 76 is coupled to a connector pin 720 at one end and a helix electrode 74 at another end; coupling means include those known to those skilled in the art, for example welding and crimping. First filar 75 is electrically isolated from second filar 76 by means of a insulative layer, for example a fluoropolymer or a polyimide coating, formed about one or both filars 75, 76, for example as illustrated for coil 63 in FIG. 4. One example of the embodiment illustrated in FIG. 7 includes sheath 74 having been formed of a polyurethane tube that is fitted over the coil and then heat re-flowed into interstices between first filar 75 and second filar 76 along intermittent lengths 700; according to alternate embodiments length 700 extends along a longer length of body 70, which may be a proximal portion, in proximity to a connector sleeve 725, or a longer portion approaching a length of lead 712 between ring electrode 72 and connector ring 718. Sheath 74 being coupled to the coil may enhance torque transfer lead body 70, which may otherwise be insufficient to fix helix 74 at an implant site without an excessive number of turns.

In the foregoing detailed description, the invention has been described with reference to specific embodiments. However, it may be appreciated that various modifications and changes can be made without departing from the scope of the invention as set forth in the appended claims.

1. A medical electrical lead, comprising:
an outer assembly comprising an insulative sheath; and
a rotating assembly extending within the outer assembly and comprising:
a connector pin terminating a proximal end of the rotating assembly,
an elongate conductor coupled to the connector pin and extending distally therefrom,
a fixation mechanism terminating a distal end of the rotating assembly and coupled to the elongate conductor within a distal portion of the outer assembly, and
a sheath overlaying the conductor and extending from the connector pin to the fixation mechanism;
wherein the rotating assembly is adapted to rotate within the outer assembly from a retracted position, wherein the fixation mechanism is enclosed within the distal portion, to an extended position, wherein the fixation mechanism extends from the distal portion, and visa versa.

2. The lead of claim 1, wherein the outer assembly further comprises a conductive structure extending from the proximal portion to the distal portion.

3. The lead of claim 2, wherein the conductive structure is formed as a coil and the outer assembly further includes:
a connector ring coupled to a proximal end of the coil and positioned on the proximal portion of the outer assembly, and
a ring electrode coupled to a distal end of the coil and positioned on the distal portion of the outer assembly.

4. The lead of claim 1, wherein the fixation mechanism includes an electrode surface.

5. The lead of claim 1, further comprising a lubricious interface between the rotating assembly and the outer assembly.

6. The lead of claim 5, wherein the lubricious interface is formed by a lubricious layer attached to an outer surface of the sheath of the rotating assembly.

7. The lead of claim 5, wherein the lubricious interface is formed by a lubricious layer attached to an inner surface of the outer assembly.

8. The lead of claim 5, wherein the lubricious interface comprises a fluoropolymer layer.

9. The lead of claim 1, wherein the sheath of the rotating assembly is fixedly coupled to the connector pin.

10. The lead of claim 1, wherein the sheath of the rotating assembly is fixedly coupled to the fixation mechanism.

11. The lead of claim 1, wherein the sheath of the rotating assembly is fixedly coupled to the elongate conductor.

12. The lead of claim 11, wherein the sheath of the rotating assembly is fixedly coupled to the elongate conductor in proximity to the connector pin.

13. The lead of claim 11, wherein the sheath of the rotating assembly is fixedly coupled to the elongate conductor in proximity to the fixation mechanism.

14. The lead of claim 11, wherein the sheath of the rotating assembly is fixedly coupled to the elongate conductor at intermittent sites along a length of the conductor.

15. The lead of claim 11, wherein the sheath of the rotating assembly is fixedly coupled to the conductor along a length approximately equal to an entire length of the conductor.

16. The lead of claim 1, wherein the sheath of the rotating assembly comprises an insulative material.

17. The lead of claim 16, wherein the insulative material is selected from the group consisting of polyurethanes, silicones, polyimides and fluoropolymers.

18. The lead of claim 1, wherein the elongate conductor is formed as a coil including one or more wire filars.

19. The lead of claim 18, wherein the coil has an outer diameter of less than approximately 0.03 inch.

20. The lead of claim 18, wherein the one or more filars include only two filars.

21. The lead of claim 20, wherein a one of the two filars includes a low-resistance core.

22. The lead of claim 21, wherein the two filars each have a diameter of less than or equal to approximately 0.006 inch.

23. The lead of claim 18, wherein the sheath of the rotating assembly is fixedly coupled to the conductor by a mechanical interlocking between the one or more wire filars.

24. The lead of claim 18, wherein the elongate conductor includes an insulative layer formed about at least one of the one or more wire filars.

25. The lead of claim 24, wherein the insulative layer comprises a fluoropolymer.

26. The lead of claim 24, wherein the insulative layer comprises a polyimide.

27. The lead of claim 1, wherein the elongate conductor is formed as a cabled bundle of wires.

28. The lead of claim 27, wherein the sheath of the rotating assembly is fixedly coupled to the conductor by mechanical interlocking between a portion of the cable bundled of wires.

29. The lead of claim 27, wherein the conductor includes an insulative layer formed about the cabled bundle.

30. The lead of claim 29, wherein the insulative layer comprises a fluoropolymer.

31. The lead of claim 29, wherein the insulative layer comprises a polyimide.

32. The lead of claim 1, wherein the fixation mechanism of the rotating assembly includes a helical fixation element, which forms a portion of the fixation mechanism that extends from the distal portion of the outer assembly in the extended position.

33. The lead of claim 32, wherein the helical fixation element includes an electrode surface.

34. The lead of claim 1, wherein the rotating assembly extends coaxially within the outer assembly.

35. The lead of claim 1, wherein the insulative sheath is a multi-lumen tube.

36. A medical electrical lead, comprising:
an elongate conductor coil; and
a sheath extending over the conductor coil;
wherein a limited portion of the conductor coil is embedded in the sheath.

37. The lead of claim 36, wherein the limited portion is in proximity to a proximal end of the lead.

38. The lead of claim 36, wherein the limited portion is in proximity to a distal end of the lead.

39. The lead of claim 36, wherein the limited portion is defined by intermittent lengths along the lead.

40. The lead of claim 36, wherein the conductor coil includes a first filar and a second filar.

41. The lead of claim 40, wherein the first filar is electrically isolated from the second filar.

42. The lead of claim 36, wherein the sheath comprises an insulative material.

43. The lead of claim 42, wherein the insulative material is selected from the group consisting of polyurethanes, silicones, polyimides and fluoropolymers.

44. The lead of claim 36, wherein the sheath is heat re-flowed to embed the limited portion of the conductor coil within the sheath.

45. The lead of claim 36, wherein the coil has an outer diameter of less than approximately 0.03 inch.

46. The lead of claim 40, wherein a one of the two filars includes a low-resistance core.

47. The lead of claim 46, wherein the two filars each have a diameter of less than or equal to approximately 0.006 inch.
